(19) United States
(12) Patent Application Publication
(10) Pub. No.: US 2010/0121133 A1
(43) Pub. Date: May 13, 2010

(54) Apparatus and methods for measuring pressure and flow in cardiac assist devices and peripheral vasculature

(76) Inventor: Douglas B. Schumer, Bala Cynwyd, PA (US)

Correspondence Address: Ballard Spahr LLP, SUITE 1000, 999 PEACHTREE STREET, ATLANTA, GA, 30309-3915, US

(21) Appl. No.: 12/291,201

(22) Filed: Nov. 7, 2008

(51) Int. Cl. A61N 1/362

(52) U.S. Cl. 600/17

(57) ABSTRACT

Disclosed are apparatus and methods for measuring pressure and/or flow in blood circulatory systems of a patient using cardiac assist devices. By way of example, a left ventricular assist device may be coupled between the left ventricle of the patient's heart and the aorta. One or more sensors, such as pressure sensors, are located at one or more selected locations in the heart, in blood-carrying conduits that are coupled to the cardiac assist device, or in the aorta, that sense pressure, for example, to monitor a patient's cardiac function. A monitoring device is coupled to the sensors that implements an algorithm that determines pressure and/or blood flow rate in the circulatory system using pressures and/or flow data derived from the sensors.

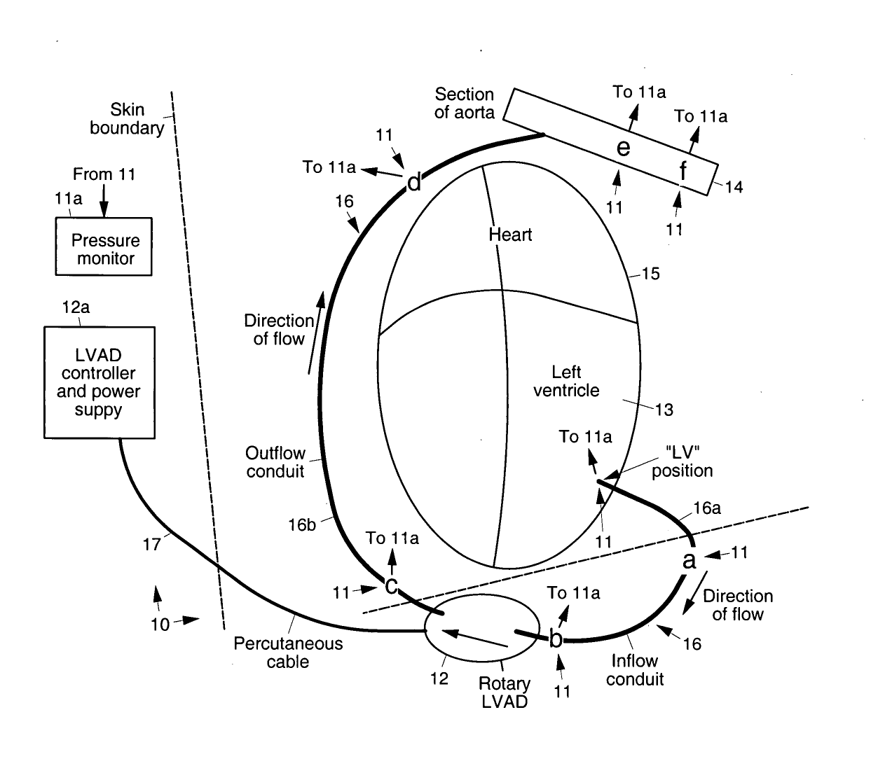

### BACKGROUND OF THE INVENTION

The present invention relates to apparatus and methods for measuring pressure and flow in cardiac assist device, such as left ventricular assist devices (LVAD), and in circulatory systems of patient's using cardiac assist devices.

For patients with conditions that compromise cardiac function, such as cardiomyopathy, for example, a cardiac assist device, such as a left ventricular assist device, is an option for treatment. In the case of end-stage NYHA Class 4 heart failure patients, the left ventricular assist device may be the only effective treatment option while the patient awaits transplant (“bridge to transplant”), or as a treatment instead of transplant (“destination therapy”).

The LVAD is a blood pump connected between the left ventricle and the aorta via blood-carrying conduits, such as is illustrated in the drawing FIGURE. The mechanical pump unloads the compromised heart by sharing the work required to provide circulation. In some cases the use of an LVAD has enabled recovery of sufficient cardiac function to permit removal of the LVAD.

Two general types of pumping mechanisms are employed in LVAD designs. First generation devices typically were based upon the use of a pulsatile pump in which a pumping chamber is passively filled by the patient's left ventricle. Upon detecting that the chamber is full, the pump actuates, compressing the LVAD chamber, providing the forceful ejection of the blood to the body, directed via unidirectional valves to control forward flow. Second generation LVADs employ smaller continuously running efficient rotary pump motor mechanisms.

With rotary pumps, motor speed controls blood flow. Unlike a passively filled pulsatile pump, there is no intrinsic way to ascertain the degree to which the heart is being emptied by the auxiliary mechanical pump. This is also the case with a class of pulsatile pumps that employ an active pumping mechanism for moving blood from a heart chamber, but for specificity in the examples described herein, a rotary pump is used for illustration. Without feedback as to the degree or rate of emptying the chamber, a too-slow pump may under-deliver pumping assistance, and an overly aggressive pump speed can collapse the heart chamber, with the potential of initiating undesirable arrhythmias. At present, algorithms based upon such indirect measures as motor speed and power consumption are used to derive information about pressure and flow, and inferentially about the degree of ventricular (or other chamber) emptying.

It would therefore be desirable to have a more direct measure of fluid pressure and flow in the LVAD/conduit system, and nearby circulatory system, to (1) provide information to monitor and control the device performance, (2) monitor the patient's physiologic parameters in real-time, and (3) monitor trends in the patient's cardiac function.

### BRIEF DESCRIPTION OF THE DRAWINGS

The features and advantages of the present invention may be more readily understood with reference to the following detailed description taken in conjunction with the accompanying drawing FIGURE, wherein like reference numerals designate like structural elements, and which is a schematic representation of an implanted pressure/flow monitoring apparatus comprising a left ventricular assist device, not drawn to size or proportion.

### DETAILED DESCRIPTION OF THE INVENTION

Referring now to the sole drawing FIGURE, disclosed are measurement systems and apparatus comprising a cardiac assist device 10, such as a ventricular assist device 10, and in particular, a left ventricular assist device (LVAD) 10, and one or more sensors 11, and in particular, pressure sensors 11. The LVAD 10 comprises a blood pump 12 connected between the left ventricle 13 and aorta 14 of a heart 15 via blood-carrying conduits 16, referred to as an inflow conduit 16a and an outflow conduit 16b. The LVAD 10 comprises an external console 12a that includes a controller and power supply coupled to the blood pump 12 via a percutaneous cable 17. The apparatus also comprises a pressure monitor 11a that is coupled in a wired or wireless fashion to the one or more sensors 11 (pressure sensors 11). The external console 12a or the pressure monitor 11a may comprise an interrogator, for example, that is used to poll the sensors 11 to extract data, such as pressure data, therefrom.

Exemplary LVADs 10 include those that are internally implanted and those that are external to the body. In the case of implanted devices 10, control of the pump 12, and power, derived from a battery or compressed air, is provided by the external console 12a, connected to the implanted pump 12 via the permanent percutaneous cable 17. In the case of externally worn pumps 12, percutaneous blood-carrying conduits 16a, 16b are necessary for connecting the pump 12 to the aorta 14, and connecting the pump 12 to the ventricular cannula 16a, which comprises the pump inflow conduit 16a.

The pressure sensors 11 may be used to sense local blood pressure and determine fluid flow rates. Exemplary apparatus, comprising a pressure/flow monitoring LVADs 10 may include pressure sensors 11 at one or more of the following locations of the LVAD system and/or patient. With reference to the drawing FIGURE, these locations are:

(a) inside the inflow conduit 16a of the LVAD 10,

(b) at a second location in the inflow conduit 16a,

(c) inside the outflow conduit 16b,

(d) at a second location in the outflow conduit 16b,

(e) at a location within the aorta 14, external to the outflow conduit 16b, or

(F) at a second location within the aorta 14.

Additionally or singularly, a pressure sensor 11 placed within the chamber of the heart 15, affixed to a LVAD conduit 16 or cannula 16 at a location designated “LV” position in the drawing FIGURE, provides a direct measure of chamber pressure, and is valuable in providing information to assess the patient's cardiac condition.

While the drawing FIGURE illustrates the LVAD 10 in the context of an internally implanted pump 12, one skilled in the art will readily understand how to implement the LVAD 10 with a pump 12 that is located exterior to the patient's body, connected via conduits 16 to a patient's heart 15 and circulatory system. Embodiments of the apparatus LVAD 10 may be enabled by wired and/or wireless pressure sensors 11 developed by the assignee of the present invention, discussed in patent applications mentioned below.

Furthermore, while this description refers to LVADs 10, the concepts disclosed herein are equally applicable to similar cardiac assist devices 10, and include those that assist right heart pumping, both atrial or ventricular, left atrial or ventricular assist devices, and multi-chamber devices or combination of devices, such as BiVAD systems, for example. For example, a right ventricular assist device receives bblood from the right ventricle and delivers it to the pulmonary artery.

Sensors 11 suited to enable the LVAD 10 include pressure sensors 11 that operate via direct electrical communication, wirelessly via radiofrequency (RF) communication and wirelessly via acoustic means. U.S. patent application Ser. No. 10/943,772 filed Sep. 16, 2004, U.S. patent application Ser. No. 11/314,046 filed Dec. 20, 2005, and U.S. patent application Ser. No. 11/157,375, filed Jun. 21, 2005 disclose pressure sensors 11 that are suited for use with the LVAD 10, and the contents of these applications are incorporated herein by reference in their entirety. In particular, the disclosure contained in these applications relating to sensors and how to make and use those sensors is incorporated herein by reference.

Used singly at a location selected from (a) inside an inflow conduit 16a of the LVAD 10, (b) at a second location in the inflow conduit 16a, (c) inside an outflow conduit 16b, or (d) at a second location in the outflow conduit, 16b, as shown in the drawing FIGURE, the real-time pressure detected by a pressure sensor 11 may be used to monitor functioning of the mechanical pump 12 and control the speed of the mechanical pump 12. Algorithms implemented in the external console 12a, or pressure monitor 11a, for example, that further refine the pressure/flow information may make use of pump motor speed and/or power as well. The sensors 11 may be wireless sensors communicating information to an internal or external electronic circuit in the external console 12a or pressure monitor 11a, or sensors 11 that communicate to internal or external control circuitry in the external console 12a or pressure monitor 11a via wires. Such wires may be designed to run through, along, or integral to, the conduits 16 required for the LVAD 10, although such routing of the wiring is not essential to the concept of the measurement systems described herein.

Sensors 11 in combination at two or more locations selected from (a) inside an inflow conduit of the LVAD, (b) at a second location in the inflow conduit, (c) inside an outflow conduit, or (d) at a second location in the outflow conduit, may provide not only pressure information, but also flow information, enabling better device monitoring and control. Flow information may be inferred by differential pressure by measurement of pressures at two points in the fluid flow path.

Flow through the aorta 15 has a contribution not only from that provided by the LVAD 10, but also fluid from the intact heart 15 pumping in parallel. Thus, a sensor 11 (e) at a location within the aorta, external to the LVAD outflow conduit, and/or (f) at a second location within the aorta 15, in conjunction with one or more sensors 11 in any of locations (a), (b), (c) or (d), may be used to infer how much load sharing is being provided by the LVAD 10. Load sharing is a measure of how much work the patient's heart 15 is providing compared to that of the LVAD 10. This flow information is useful not only on a real-time basis, but may be valuable for obtaining trend data for the purpose of guiding therapy directed at affecting cardiac recovery.

Regarding manufacture of the pressure-sensing LVAD 10, biocompatible adhesive can be used to fix the sensors 11 at locations within the LVAD 10. Alternatively, the sensors 11 may be anchored in desired locations via mechanical means such as hooks, barbs or wire basket or stent-like devices. Many other methods of incorporation are feasible and should be evident to one skilled in the art.

Thus, cardiac assist devices incorporating sensors, including pressure sensors, and related methods have been disclosed. It is to be understood that the above-described embodiments are merely illustrative of some of the many specific embodiments that represent applications of the principles discussed above. Clearly, numerous and other arrangements, including other types of sensors, can be readily devised by those skilled in the art without departing from the scope of the invention.

What is claimed is:

1. Apparatus comprising:
a cardiac assist device coupled via blood-carrying conduits between a selected chamber of a patient's heart and a selected portion of the patient's blood circulatory system;
one or more sensors disposed in one or more of the blood-carrying conduits and/or a selected location in the patient's blood circulatory system that sense pressure and/or flow in the conduits and/or blood circulatory system; and
a monitoring device coupled to the one or more sensors for determining pressure and/or blood flow in the blood circulatory system using pressures and/or flow rates derived from the one or more sensors.

2. The apparatus recited in claim 1 wherein the cardiac assist device comprises a left ventricular assist device that is coupled between the left ventricle of the patient's heart and the aorta.

3. The apparatus recited in claim 1 wherein the cardiac assist device comprises a right ventricular assist device that is coupled between the right ventricle of the patient's heart and the pulmonary artery.

4. The apparatus recited in claim 1 wherein the one or more sensors comprise pressure sensors.

5. The apparatus recited in claim 1 wherein the cardiac assist device comprises a rotary ventricular assist device.

6. The apparatus recited in claim 1 wherein the cardiac assist device comprises a multi-chamber assist device.

7. The apparatus recited in claim 1 wherein the cardiac assist device comprises an atrial assist device.

8. The apparatus recited in claim 1 wherein the one or more sensors are selectively disposed inside an inflow conduit of the cardiac assist device, at a second location in the inflow conduit, inside an outflow conduit of the cardiac assist device, at a second location in the outflow conduit, at a location within the aorta and external to the outflow conduit, at a second location within the aorta, or within a chamber of the heart adjacent to an input end of the inflow conduit.

9. Apparatus comprising:
a left ventricular assist device coupled via blood-carrying conduits between the left ventricle of a patient's heart and the aorta;
one or more pressure sensors disposed in one or more of the blood-carrying conduits and/or the aorta that sense pressure and/or flow in the patient's blood circulatory system; and
a monitoring device coupled to the one or more sensors for determining pressure and/or blood flow in the blood circulatory system using pressures and/or flow rates derived from the one or more sensors.

10. A method comprising:
coupling a cardiac assist device coupled via blood-carrying conduits between a selected chamber of a patient's heart and a selected portion of the patient's blood circulatory system;
disposing one or more sensors in one or more of the blood-carrying conduits and/or a selected location in the patient's blood circulatory system; and
determining pressure and/or flow in the patient's blood circulatory system using pressures and/or flow rates derived from the one or more sensors.

11. The method recited in claim 10 wherein the cardiac assist device comprises a left ventricular assist device that is coupled between the left ventricle of the patient's heart and the aorta.

12. The method recited in claim 10 wherein the cardiac assist device comprises a right ventricular assist device that is coupled between the right ventricle of the patient's heart and the pulmonary artery.

13. The method recited in claim 10 wherein the one or more sensors are selectively disposed inside an inflow conduit of the cardiac assist device, at a second location in the inflow conduit, inside an outflow conduit of the cardiac assist device, at a second location in the outflow conduit, at a location within the aorta and external to the outflow conduit, at a second location within the aorta, or within a chamber of the heart adjacent to an input end of the inflow conduit.
